(19) United States
(12) Patent Application Publication
(10) Pub. No.: US 2026/0097405 A1
(43) Pub. Date: Apr. 9, 2026

(54) WIRELESS AIR DUCT DEVICE AND GARBAGE DISPOSER

(71) Applicant: Guangdong Shihao Intelligent Technology Co. , Ltd., Foshan (CN)

(72) Inventors: Chuanliang Zheng, Foshan (CN); Jianwei Liu, Foshan (CN); Qing Xiao, Foshan (CN); Wennian Li, Foshan (CN); Tao Zheng, Foshan (CN)

(21) Appl. No.: 19/086,764

(22) Filed: Mar. 21, 2025

(30) Foreign Application Priority Data
Oct. 8, 2024 (CN) 202422422967.0

(51) Int. Cl. B02C 23/00; A61L 9/014

(52) U.S. Cl. CPC B02C 23/00; A61L 9/014; A61L 2209/22; B02C 2201/06

(57) ABSTRACT

Provided are a wireless air duct device and a garbage disposer, the wireless air duct device comprises an air duct body, a top cover plate, a centrifugal turbine, a motor, a seat plate, a wireless power supply receiving module, and a wireless power supply transmitting module, the air duct body comprises an air inlet cavity, an air inlet interface, an air guide cavity, an air exhaust cavity, a coaming plate, and a side cover plate, and the coaming plate is provided at a communication point between the air exhaust cavity and the air guide cavity. The wireless power supply receiving module and the wireless power supply transmitting module are provided to be conveniently assembled and disassembled with the garbage disposer. The wireless power supply receiving module is waterproof and anti-pollution, is convenient to clean, maintains power supply performance; and fast electrical connection is achieved, and the service life is long.

### CROSS-REFERENCE TO RELATED APPLICATIONS

This is a U.S. patent application which claims the priority and benefit of Chinese Patent Application Number 202422422967.0, filed on Oct. 8, 2024, the disclosure of which is incorporated herein by reference in its entirety.

### TECHNICAL FIELD

The present invention relates to a garbage disposer, especially an air exhaust device.

### BACKGROUND

According to current existing technologies, a Chinese Patent 202120598363.5 provides a kitchen garbage disposer, comprising: a machine body and a cover body covered on the machine body, wherein a disposal barrel is provided in the machine body, and an air inlet and an air outlet are also formed in an outer wall of the machine body; a hot air assembly, wherein the hot air assembly comprises an air duct provided in the machine body, the air duct is communicated with the inside of the disposal barrel, an air inlet end of the air duct is communicated with the air inlet, a heating body and an air exhaust fan are provided in the air duct, and an air outlet end of the air duct is communicated with the air outlet; and a purifying assembly, wherein the purifying assembly is provided in the air duct and is used for performing purifying treatment on a gas discharged from the disposal barrel. The kitchen garbage disposer has the problems that a power supply wire is required, and the air duct is inconvenient to assemble and disassemble.

### SUMMARY

The objective of the present invention is to provide a wireless air duct device, which has high sealing performance, adopts a wireless connection method for power supply, and is convenient to assemble and disassemble.

The present invention is implemented as follows: a wireless air duct device, which is characterized by comprising: an air duct body, a top cover plate, a centrifugal turbine, a motor, a seat plate, a wireless power supply receiving module, and a wireless power supply transmitting module,
wherein the air duct body comprises an air inlet cavity, an air inlet interface, an air guide cavity, an air exhaust cavity with a side opening, a coaming plate, and a side cover plate, the air guide cavity and the air exhaust cavity are provided on left and right sides or on front and rear sides and are communicated, and the coaming plate is provided at a communication point between the air exhaust cavity and the air guide cavity; the air inlet cavity, the air inlet interface, the air guide cavity, the air exhaust cavity and the coaming plate are made by integral molding;

the top cover plate is fastened to a top of the air inlet cavity, the motor is fixed on the seat plate, and the centrifugal turbine is fixed to a motor shaft; the seat plate serves as a rear volute plate to close the coaming plate; and the side cover plate closes and seals the side opening of the air exhaust cavity, and the wireless power supply receiving module is provided in a sealed space formed by the side cover plate and the seat plate and is connected to the motor.

The wireless air duct device is characterized in that a power supply cavity with an inner side opening is provided on the side cover plate, and the wireless power supply receiving module is provided in the power supply cavity.

The wireless air duct device is characterized in that the power supply cavity is opposite to the motor.

The wireless air duct device is characterized in that an air inlet fence is provided on the air inlet interface.

The wireless air duct device is characterized in that the air guide cavity and the air exhaust cavity are staggered.

A garbage disposer is characterized by comprising: a casing, a drying and crushing device, a drying cavity cover, an air exhaust cover, a filtering and deodorizing device, and the wireless air duct device as mentioned above,
wherein the casing comprises a crushing device cavity with an upper opening, an air exhaust device cavity with an upper opening, and an air guide channel,
the filtering and deodorizing device comprises a water receiving box and an activated carbon deodorizing box, and the activated carbon deodorizing box is communicated with the water receiving box and is detachable;
the drying and crushing device is provided in the crushing device cavity, and the drying cavity cover is hinged to the casing and covers the crushing device cavity; the wireless air duct device is communicated with the water receiving box and is detachable, the wireless air duct device and the air exhaust device cavity are detachable, the air inlet interface is communicated with the air guide channel, and the wireless power supply transmitting module is provided in the crushing device cavity and is opposite to the wireless power supply receiving module; and the activated carbon deodorizing box is communicated with the air exhaust cover and is detachable, and the air exhaust cover covers the upper opening of the air exhaust device cavity and is detachable.

The garbage disposer is characterized in that the water receiving box is detachably connected to the air exhaust device cavity of the casing.

The garbage disposer is characterized in that the wireless power supply transmitting module is provided on a partition between the crushing device cavity and the air exhaust device cavity.

The garbage disposer is characterized in that the air exhaust cover is provided with an interface cavity with a lower opening, and an air exhaust fence is provided on a top plate of the interface cavity;
and the activated carbon deodorizing box is inserted into and detachably fitted with the interface cavity.

The garbage disposer is characterized in that the water receiving box has an upper opening,
and further comprises an inlet and exhaust cover,
an air inlet seat and an air exhaust hole are provided on the inlet and exhaust cover, the air inlet seat is provided with an inner air inlet interface, and an inner air exhaust interface is provided on the air exhaust hole;
and the inlet and exhaust cover is provided on the water receiving box and is fixed, the wireless air duct device is plugged into the inner air inlet interface, and an air inlet end of the activated carbon deodorizing box is plugged into the inner air exhaust interface.

According to the wireless air duct device of the present invention, the wireless power supply receiving module and the wireless power supply transmitting module are provided to be conveniently assembled and disassembled with the garbage disposer; during use or cleaning, the wireless power supply receiving module is waterproof and anti-pollution, is convenient to clean, and maintains power supply performance; and fast electrical connection is achieved, and the service life is long.

### BRIEF DESCRIPTION OF DRAWINGS

FIG. 1 is a stereoscopic exploded view of the present invention.

FIG. 2 is a section view of the present invention.

FIG. 3 is a perspective view of the present invention.

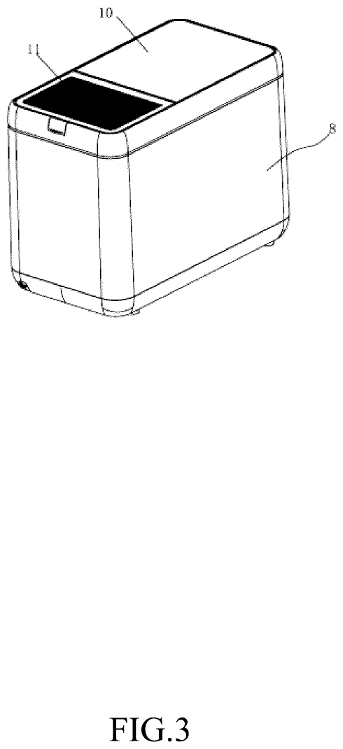

FIG. 4 is a partial enlarged view of FIG. 1.

FIG. 5 is a first stereoscopic exploded view of the present invention.

FIG. 6 is a second stereoscopic exploded view of the present invention.

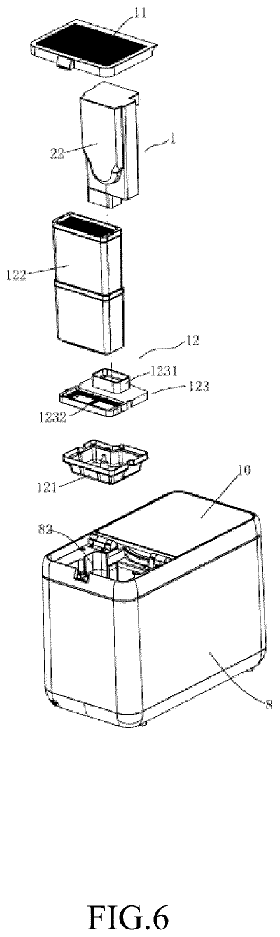

### DESCRIPTION OF EMBODIMENTS

Embodiments of the present invention are described in detail below, and examples of the embodiments are illustrated in the drawings, in which same or similar reference numerals throughout represent same or similar elements or elements with same or similar functions. The embodiments described below with reference to the drawings are exemplary, which are only used to explain the present invention and cannot be understood as limiting the present invention.

In the description of the present invention, it should be noted that, unless otherwise clearly stated and limited, terms, such as “mount”, “connect”, and “connection” should be understood in a broad sense, for example, may be fixed connection, and may also be detachable connection, or integral connection; and may be direct connection, and may also be indirect connection through an intermediate medium, or internal communication between two elements. For those of ordinary skill in the art, the above terms may be understood to have specific meanings in the present invention according to specific circumstances.

### Embodiment 1

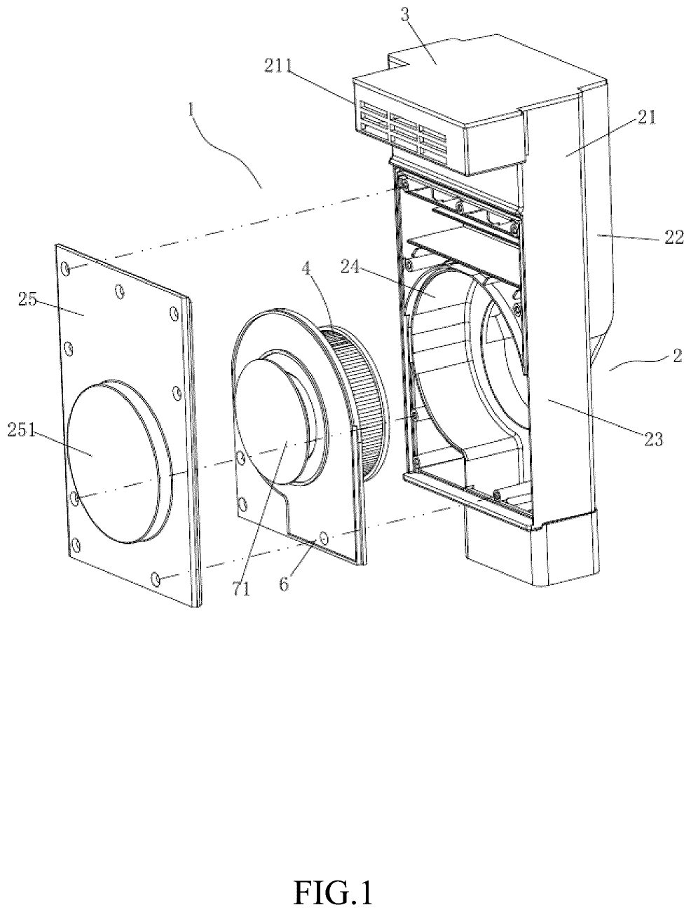

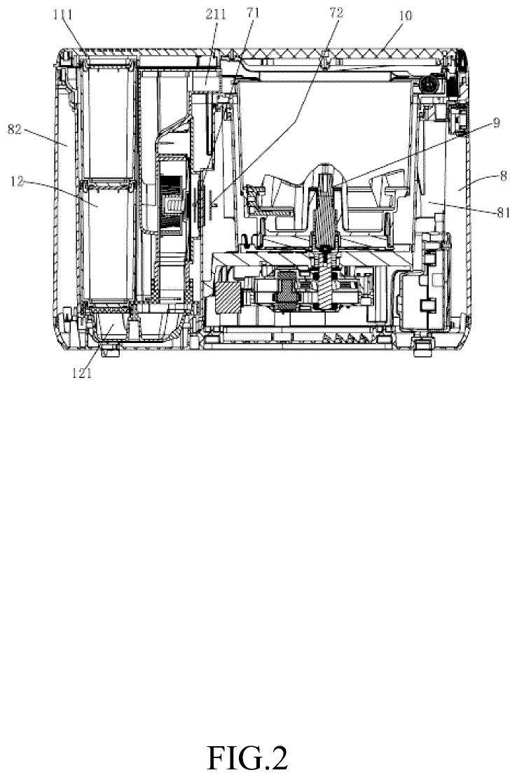

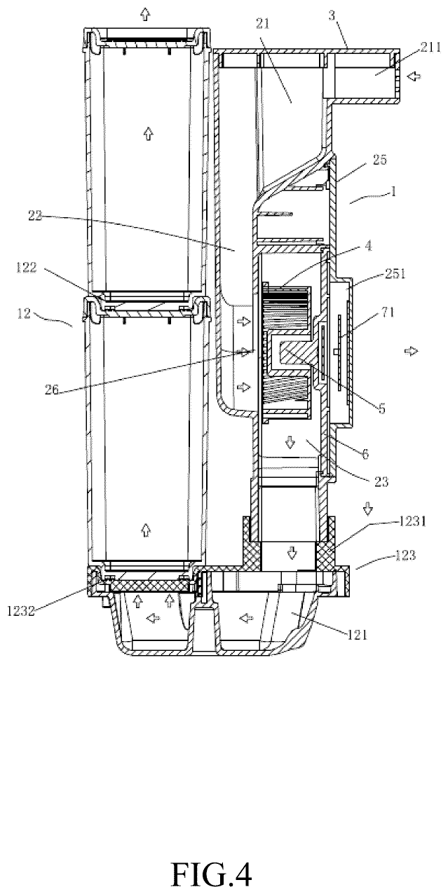

As shown in FIG. 1, FIG. 2 and FIG. 4, a wireless air duct device 1 comprises an air duct body 2, a top cover plate 3, a centrifugal turbine 4, a motor 5, a seat plate 6, a wireless power supply receiving module 71, and a wireless power supply transmitting module 72,
the air duct body 2 comprises an air inlet cavity 21, an air inlet interface 211 located in the air inlet cavity 21, an air guide cavity 22, an air exhaust cavity 23 with a side opening, a coaming plate 24, and a side cover plate 25, the air guide cavity 22 and the air exhaust cavity 23 are provided on left and right sides or on front and rear sides and are communicated, and a communication point is provided with a flow guide hole 16; the coaming plate 24 is provided at the communication point between the air exhaust cavity 23 and the air guide cavity 22; a wall plate of the air exhaust cavity 23 that is opposite to the coaming plate 24 serves as a front volute plate, and the flow guide hole serves as an air inlet hole; the air inlet cavity 21, the air inlet interface 211, the air guide cavity 22, the air exhaust cavity 23 and the coaming plate 24 are made by integral molding;
the top cover plate 3 is fastened to a top of the air inlet cavity 21, the motor 5 is fixed on the seat plate 6, and the centrifugal turbine 4 is fixed to a motor shaft; the seat plate 6 serves as a rear volute plate to close an opening on one side of the coaming plate 24; the side cover plate 25 closes and seals the side opening of the air exhaust cavity 23, and the wireless power supply receiving module 71 is provided in a sealed space formed by the side cover plate 25 and the seat plate 6 and is connected to the motor; during use or cleaning, the wireless power supply receiving module is waterproof and anti-pollution, is convenient to clean, and maintains power supply performance;
the flow guide hole 26 is located at a lower end of the air guide cavity 22; and by adopting such structure, the wireless air duct device 1 is convenient to assemble and disassemble on a garbage disposer, in particular, electrical connection can be conveniently achieved, and the service life is long.

As a further improvement of the present invention: a power supply cavity 251 with an inner side opening is provided on the side cover plate 25, and the wireless power supply receiving module 71 is provided in the power supply cavity 251.

The power supply cavity 251 is opposite to the motor 5.

As a further improvement of the present invention: an air inlet fence is provided on the air inlet interface 211.

As a further improvement of the present invention: the air guide cavity 22 and the air exhaust cavity 23 are staggered.

### Embodiment 2

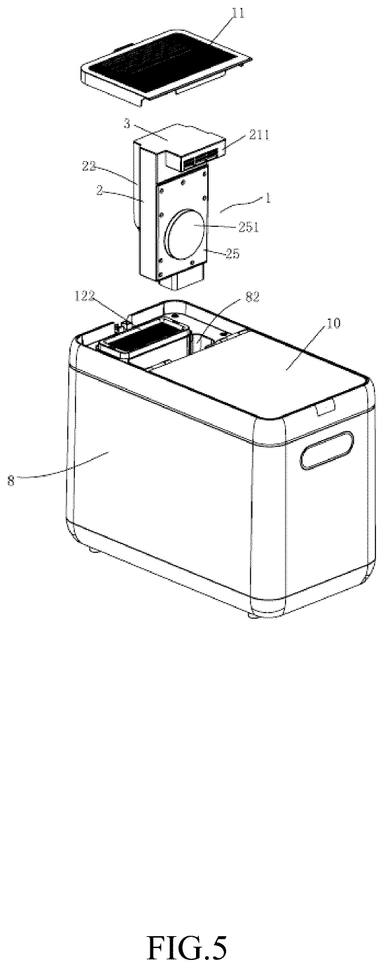

As shown in FIG. 2 and FIG. 5, a garbage disposer comprises a casing 8, a drying and crushing device 9, a drying cavity cover 10, an air exhaust cover 11, a filtering and deodorizing device 12, and a wireless air duct device 1,
the casing 8 comprises a crushing device cavity 81 with an upper opening, an air exhaust device cavity 82 with an upper opening, and an air guide channel (not marked), and the air guide channel communicates the crushing device cavity 81 with the air exhaust device cavity 82;
the filtering and deodorizing device 12 comprises a water receiving box 121 and an activated carbon deodorizing box 122, and the activated carbon deodorizing box 122 is communicated with the water receiving box 121 and is detachable;
the drying and crushing device 9 is provided in the crushing device cavity 81, and the drying cavity cover 10 is hinged to the casing 8 and covers the upper opening of the crushing device cavity 81; the wireless air duct device 1 is communicated with the water receiving box 121 and is detachable, the wireless air duct device 1 and the air exhaust device cavity 82 are detachable, the air inlet interface 211 is communicated with the air guide channel, and the wireless power supply transmitting module 72 is provided in the crushing device cavity 81 and is opposite to the wireless power supply receiving module 71; and the activated carbon deodorizing box 122 is communicated with the air exhaust cover 11 and is detachable, and the air exhaust cover 11 covers the upper opening of the air exhaust device cavity 82 and is detachable.

The water receiving box 121 is detachably connected to the air exhaust device cavity 82 of the casing 8.

The wireless power supply transmitting module 72 is provided on a partition between the crushing device cavity 81 and the air exhaust device cavity 82 and is located in the drying and crushing device 9.

As a further improvement of the present invention: the air exhaust cover 11 is provided with an interface cavity 111 with a lower opening, and an air exhaust fence is provided on a top plate of the interface cavity 111;
and the activated carbon deodorizing box 122 is inserted into and detachably fitted with the interface cavity 111.

As a further improvement of the present invention: the water receiving box 121 has an upper opening,
and further comprises an inlet and exhaust cover 13,
an air inlet seat 1231 and an air exhaust hole 1232 are provided on the inlet and exhaust cover 123, the air inlet seat 1231 is provided with an inner air inlet interface, and an inner air exhaust interface is provided on the air exhaust hole 1232;
and the inlet and exhaust cover 123 is provided on the water receiving box 121 to cover the upper opening of the water receiving box 121 and is fixed, the wireless air duct device 1 is plugged into the inner air inlet interface, and an air inlet end of the activated carbon deodorizing box 122 is plugged into the inner air exhaust interface.

Although the embodiments of the present invention have been shown and described, it may be understood by those of ordinary skill in the art that various changes, modifications, substitutions and alternations of the embodiments may be made without departing from the principles and spirit of the present invention, and the scope of the present invention is defined by the claims and equivalents thereof.

What is claimed is:

1. A wireless air duct device, comprising: an air duct body, a top cover plate, a centrifugal turbine, a motor, a seat plate, a wireless power supply receiving module, and a wireless power supply transmitting module,
wherein the air duct body comprises an air inlet cavity, an air inlet interface, an air guide cavity, an air exhaust cavity with a side opening, a coaming plate, and a side cover plate, the air guide cavity and the air exhaust cavity are provided on left and right sides or on front and rear sides and are communicated, and the coaming plate is provided at a communication point between the air exhaust cavity and the air guide cavity; the air inlet cavity, the air inlet interface, the air guide cavity, the air exhaust cavity and the coaming plate are made by integral molding;
the top cover plate is fastened to a top of the air inlet cavity, the motor is fixed on the seat plate, and the centrifugal turbine is fixed to a motor shaft; the seat plate serves as a rear volute plate to close the coaming plate; and the side cover plate closes and seals the side opening of the air exhaust cavity, and the wireless power supply receiving module is provided in a sealed space formed by the side cover plate and the seat plate and is connected to the motor.

2. The wireless air duct device according to claim 1, wherein a power supply cavity with an inner side opening is provided on the side cover plate, and the wireless power supply receiving module is provided in the power supply cavity.

3. The wireless air duct device according to claim 2, wherein the power supply cavity is opposite to the motor.

4. The wireless air duct device according to claim 1, wherein an air inlet fence is provided on the air inlet interface.

5. The wireless air duct device according to claim 1, wherein the air guide cavity and the air exhaust cavity are staggered.

6. A garbage disposer, comprising: a casing, a drying and crushing device, a drying cavity cover, an air exhaust cover, a filtering and deodorizing device, and the wireless air duct device according to claim 1,
wherein the casing comprises a crushing device cavity with an upper opening, an air exhaust device cavity with an upper opening, and an air guide channel,
the filtering and deodorizing device comprises a water receiving box and an activated carbon deodorizing box, and the activated carbon deodorizing box is communicated with the water receiving box and is detachable;
the drying and crushing device is provided in the crushing device cavity, and the drying cavity cover is hinged to the casing and covers the crushing device cavity; the wireless air duct device is communicated with the water receiving box and is detachable, the wireless air duct device and the air exhaust device cavity are detachable, the air inlet interface is communicated with the air guide channel, and the wireless power supply transmitting module is provided in the crushing device cavity and is opposite to the wireless power supply receiving module; and the activated carbon deodorizing box is communicated with the air exhaust cover and is detachable, and the air exhaust cover covers the upper opening of the air exhaust device cavity and is detachable.

7. The garbage disposer according to claim 6, wherein the water receiving box is detachably connected to the air exhaust device cavity of the casing.

8. The garbage disposer according to claim 6, wherein the wireless power supply transmitting module is provided on a partition between the crushing device cavity and the air exhaust device cavity.

9. The garbage disposer according to claim 6, wherein the air exhaust cover is provided with an interface cavity with a lower opening, and an air exhaust fence is provided on a top plate of the interface cavity;
and the activated carbon deodorizing box is inserted into and detachably fitted with the interface cavity.

10. The garbage disposer according to claim 6, wherein the water receiving box has an upper opening,
and further comprises an inlet and exhaust cover,
an air inlet seat and an air exhaust hole are provided on the inlet and exhaust cover, the air inlet seat is provided with an inner air inlet interface, and the air exhaust hole is provided with an inner air exhaust interface;
and the inlet and exhaust cover is provided on the water receiving box and is fixed, the wireless air duct device is plugged into the inner air inlet interface, and an air inlet end of the activated carbon deodorizing box is plugged into the inner air exhaust interface.
